(19) United States
(12) Patent Application Publication
(10) Pub. No.: US 2023/0095498 A1
(43) Pub. Date: Mar. 30, 2023

(54) NOVEL COMPOUND AND USE THEREOF

(71) Applicant: THE CATHOLIC UNIVERSITY OF KOREA INDUSTRY-ACADEMIC COOPERATION FOUNDATION, Seoul (KR)

(72) Inventors: Chul-Woov YANG, Seou (KR); Dong-Yun SHIN, Seoul (KR); Mi-La CHO, Seoul (KR); Seon-Yeong LEE, Seoul (KR); Sun-Woo LIM, Seoul (KR); Yoo-Jin SHIN, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 17/794,823

(22) PCT Filed: Jan. 14, 2021
(86) PCT No.: PCT/KR2021/000555

§ 371 Date: Jul. 22, 2022

(30) Foreign Application Priority Data
Jan. 22, 2020 (KR) 10-2020-0008356

(51) Int. Cl. C07D 401/14; A61P 13/12; A61P 29/00

(52) U.S. Cl. CPC C07D 401/14; A61P 13/12; A61P 29/00

(57) ABSTRACT

Provided is a novel compound and use thereof which has an excellent immunological regulation ability while not exhibiting toxicity in vivo, and has no nephrotoxicity and rather has a kidney protective effect, and thus can be used for the treatment of immune diseases such as autoimmune diseases caused by abnormal regulation of various immune responses, inflammatory diseases, and transplant rejection diseases. A transplant rejection avatar animal model was confirmed to be humanized, by confirming an increase in serum creatinine, an indicator of transplant rejection in a patient, an increase in human CD4-positive cells, and infiltration of inflammatory cytokine IL-17 into kidney tissue of the animal model. It was confirmed that, when an immunosuppressant is administered, the increased serum creatinine, human CD4-positive cells, and the infiltration of inflammatory cytokine IL-17 are reduced, and thus an animal model, in which the immune status of a patient is reflected, is effectively constructed.

### TECHNICAL FIELD

The present invention relates to a novel compound and its use for the treatment of immune diseases.

### BACKGROUND ART

An immune disease is a disease in which components of the mammalian immune system cause, mediate, or otherwise contribute to the pathological conditions of the mammals, and particularly, inflammatory disorder is one of the most important health problems around the world. Inflammation is a generally localized protective response of body tissues to the host intrusion by external substances or harmful stimuli. The cause of inflammation may be a state associated with infectious causes such as bacteria, viruses, and parasites; physical causes such as burns or radiation; chemicals such as toxins, drugs, or industrial agents; the immune responses such as allergy and autoimmune responses; or oxidative stress.

The inflammation is characterized by pain, a red phenomenon, swelling, heat, and an eventual functional loss of an infected area. These symptoms are results of a series of complex interactions occurring between cells in the immune system. As a result, due to the response of the cells, an interaction network of inflammatory mediators in many groups is generated: a protein (for example, cytokines, enzymes (e.g., protease, peroxidase), a major basic protein, adhesive molecules (ICAM, VCAM), lipid mediators (e.g., eicosanoid, prostaglandin, leukotriene, platelet activating factor (PAF)), reactive oxygen species (e.g., hydroperoxide, superoxide anion 02-, nitric oxide (NO), etc.). However, most of the mediators of the inflammation are also normal cell activity regulators. Accordingly, while the host is not controlled due to the lack of the inflammatory response, the host is damaged (that is, inflected), and therefore, due to the chronic inflammation, partially, some of the aforementioned mediators are excessively generated and the mediated inflammatory diseases are caused.

Further, an autoimmune disease which is one of the immune diseases has a feature that the immune system causes a spontaneous response by attacking its organ. The responses are caused by recognition of autoantigen by the T lymphocytes, resulting in humoral (production of auto-antigens) and cellular (increase of cytotoxic activity of lymphocytes and macrophages) immune responses. The autoimmune diseases may include diseases below, rheumatic diseases, psoriasis, systemic dermatomyositis, multiple sclerosis, lupus erythematosus, deterioration of immune responses by antigens, i.e., asthma, drug or food allergies, etc. The diseases are limitative and chronic diseases, and in some cases, fatal, and until now, an effective treatment method capable of treating the diseases is not present. Therefore, drugs, medicines, or media capable of reducing or alleviating the diseases in the progress of the corresponding disease may become an important solved means for a patient's health.

Meanwhile, transplantation refers to the process of taking cells, tissues, or organs (i.e., grafts) from one subject and transferring them to another subject. The subject who provides the grafts is called a donor and the subject who receives the grafts is called a recipient or a host. In the case of transplanted organs, rejection occurs due to an immunological response to the histocompatibility antigens (transplanted antigens) on the cell surface of the grafts. The long-term engraftment of grafts in a recipient who is not immune-suppressed is limited to the case in which histocompatibility is completely or mostly consistent, and thus the genetic relationship between the donor and the recipient is a factor that greatly influences the engraftment period of the grafts. In general, rejections rarely occur in autografts and isografts; however, rejections occur almost in allografts.

For successful organ transplantation, a recipient's immune rejection to cells and organs to be transplanted needs to be overcome.

The major mediators of the immune rejection in transplantation are T cells and a major histocompatibility complex (MHC) which is expressed in a graft is recognized by a T cell receptor and the immune response is induced and the transplantation rejection occurs.

Although the success rate of transplantation has risen recently with the improvement of surgical procedures and HLA typing techniques and the development of immunosuppressants, the death rate due to immune rejection and the side effects of the immunosuppressants is still high. Thus, there is a demand for the development of a novel effective and safe immunosuppressant. The common object of all the conventional immunosuppressants is to suppress T cell-mediated immunity to a graft. In order to suppress T cell-mediated acute rejection after clinical transplant, a nonspecific immunosuppressant is administered every day (Pirsch, J. D., curr. opin. organ. transplant., 2, 76 to 81, 1997). Examples of immunosuppressants generally used include azathioprine and mycophenolate mofetil that contain glucocorticosteroids to inhibit DNA synthesis and thus to suppress T cell proliferation, cyclosporine A and tacrolimus as calcineurin inhibitors, and the like.

These drugs have been improved a lot in overcoming an immune rejection of an organ transplantation patient, but they have problems including a temporary therapeutic effect and high toxicity. Therefore, although it is important to develop an immunosuppressant for suppressing transplantation rejection, at this point in time when an immunosuppressant having a clear therapeutic effect needs to be further developed, the most effective and fastest method for minimizing side effects caused by administration of an immunosuppressant is to administer an immunosuppressant suitable for the patient's immune system immunosuppressant.

However, if a method is developed that may treat the immunosuppressant according to the immune status by checking the immune status in the body of the transplanted patient, it will be possible to reduce pain of the patient caused by the immunosuppressant.

Intensive efforts have been made to find suitable drugs and methods by exploring treatment methods for autoimmune diseases and transplantation rejection. Today, the treatment of autoimmune diseases and transplantation rejection is mainly based on the use of immunosuppressive drugs such as glucocorticoids, calcineurin inhibitors, and antiproliferatives-antimetabolites. However, since such pharmacological therapy acts on a variety of targets, the overall immune function may be reduced. Otherwise, when such pharmacological therapy is used for a long period of time, various cytotoxic effects become a problem, suppressing the immune system in a non-specific manner, thereby exposing a patient to the risk of infection and cancer. Because calcineurin and glucocorticoids present another problem due to their nephrotoxicity and diabetes-inducing properties, their use is limited in some clinical symptoms (for example, kidney insufficiency, diabetes, etc.).

Accordingly, it is necessary to develop a new therapeutic agent for immune diseases with excellent therapeutic effects without side effects as a substance that may treat immune diseases such as autoimmune diseases, transplantation rejection, and inflammatory diseases. In addition, it is necessary to develop a method capable of treating immunosuppressants according to the immune status by checking the immune status in the body of a patient.

### DISCLOSURE

### Technical Problem

An aspect of the present invention is directed to providing a compound represented by Formula 1 below or a pharmaceutically acceptable salt thereof.

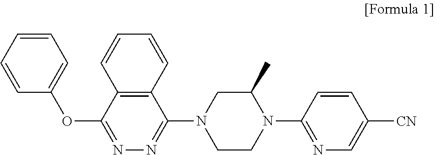 [SMILES: C[C@@H]1CN(c2nnc(Oc3ccccc3)c3ccccc23)CCN1c1ccc(C#N)cn1]

Another aspect of the present invention is directed to providing an immunosuppressant including the compound as an active ingredient.

Yet another aspect of the present invention is directed to providing a pharmaceutical composition for preventing or treating an immune disease, including the compound as an active ingredient.

Yet another aspect of the present invention is directed to providing a method for preventing or treating an immune disease, in which the method includes administering to a subject an effective amount of a compound represented by Formula 1 or a pharmaceutically acceptable salt thereof.

Yet another aspect of the present invention is directed to providing a pharmaceutical composition for preventing or treating transplantation rejection or transplantation rejection disease, including the compound as an active ingredient.

Yet another aspect of the present invention is directed to providing a method for preventing or treating transplantation rejection or transplantation rejection disease, in which the method includes administering to a subject an effective amount of a compound represented by Formula 1 or a pharmaceutically acceptable salt thereof.

### Technical Solution

An embodiment of the present invention provides a compound represented by Formula 1 below or a pharmaceutically acceptable salt thereof:

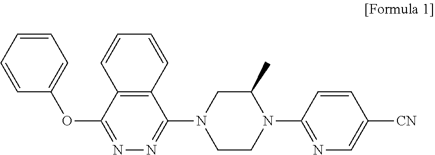 [SMILES: C[C@@H]1CN(c2nnc(Oc3ccccc3)c3ccccc23)CCN1c1ccc(C#N)cn1]

In addition, an embodiment of the present invention provides an immunosuppressant including the compound as an active ingredient.

In addition, an embodiment of the present invention provides a pharmaceutical composition for preventing or treating an immune disease, including the compound as an active ingredient.

In addition, an embodiment of the present invention provides a method for preventing or treating an immune disease, in which the method includes administering to a subject an effective amount of a compound represented by Formula 1 or a pharmaceutically acceptable salt thereof.

In addition, an embodiment of the present invention provides a pharmaceutical composition for preventing or treating transplantation rejection or transplantation rejection disease, including the compound as an active ingredient.

In addition, an embodiment of the present invention provides a method for preventing or treating transplantation rejection or transplantation rejection disease, in which the method includes administering to a subject an effective amount of a compound represented by Formula 1 or a pharmaceutically acceptable salt thereof.

### Advantageous Effects

The novel compound of an embodiment of the present invention has an excellent immunological regulation ability while not exhibiting toxicity in vivo, and has no nephrotoxicity unlike conventional immunosuppressants and rather has a kidney protective effect, and thus can be used for the treatment of immune diseases such as autoimmune diseases caused by abnormal regulation of various immune responses, inflammatory diseases, and transplantation rejection diseases. In a transplantation rejection avatar animal model of an embodiment of the present invention, an increase in serum creatinine, which is an indicator of transplantation rejection in a patient, an increase in human CD4-positive cells, and infiltration of inflammatory cytokine IL-17 into kidney tissue of the animal model were confirmed. It has also been confirmed that, when an immunosuppressant is administered, the increased serum creatinine, human CD4-positive cells, and the infiltration of inflammatory cytokine IL-17 are reduced, and thus an animal model, in which the immune status of a patient is reflected, is effectively constructed, and the consequent effect of the immunosuppressant has been confirmed. Accordingly, the novel compound can be usefully used in related industries.

### DESCRIPTION OF DRAWINGS

FIG. 1 illustrates a cytoprotective effect of an SD911 compound of an embodiment of the present invention.

FIG. 2 illustrates a result of a toxicity test on immune cells of the SD911 compound of an embodiment of the present invention.

FIG. 3 illustrates an inhibitory efficacy of ROS production in an HK-2 cell line of the SD911 compound of an embodiment of the present invention.

FIG. 4 illustrates a result of identifying the immunological regulation ability of the SD911 compound of an embodiment of the present invention.

FIG. 5 illustrates an SIP lyase activity inhibitory effect of the SD911 compound of an embodiment of the present invention.

FIG. 6A illustrates an ROS production inhibitory effect of the SD911 compound of an embodiment of the present invention in an HK-1 cell line.

FIG. 6B is quantification of ROS production in the HK-1 cell line of the SD911 compound of an embodiment of the present invention.

FIG. 7A illustrates an effect of the SD911 compound of an embodiment of the present invention on the reduction of apoptosis by tacrolimus (Tac).

FIG. 7B is quantification of an effect of the SD911 compound of an embodiment of the present invention on the reduction of apoptosis by tacrolimus.

FIG. 8 is a schematic diagram of a construction process of a mouse animal model of an embodiment of the present invention.

FIG. 9A is a diagram illustrating the analysis of engraftment of human cells by flow cytometry in the mouse model of an embodiment of the present invention.

FIG. 9B is a diagram analyzing the level of SCR in the mouse model of an embodiment of the present invention.

FIG. 10A is a diagram identifying the extent of damage to kidney tissue in a mouse model injected with normal PBMCs of an embodiment of the present invention.

FIG. 10B is a diagram identifying the extent of damage to kidney tissue in a mouse model injected with PBMCs of a transplantation rejection patient of an embodiment of the present invention.

FIG. 10C is a diagram illustrating quantification of kidney damage scores according to the treatment of the immunosuppressant SD911 in a mouse model injected with normal PBMCs and a mouse model injected with PBMCs of a transplantation rejection patient of an embodiment of the present invention.

FIG. 11A is a diagram identifying human CD4-positive cell infiltration by immunochemical histology staining in a mouse model injected with normal PBMCs of an embodiment of the present invention.

FIG. 11B is a diagram identifying human CD4-positive cell infiltration by immunochemical histology staining in a mouse model injected with PBMCs of a transplantation rejection patient of an embodiment of the present invention.

FIG. 11C is quantification of the number of CD4-positive cells according to the treatment with the immunosuppressant SD911 in a mouse model injected with normal PBMCs and a mouse model injected with PBMCs of a transplantation rejection patient of an embodiment of the present invention.

FIG. 12A is a diagram identifying the infiltration of IL-17-positive cells by immunochemical histology staining in a mouse model injected with normal PBMCs of an embodiment of the present invention.

FIG. 12B is a diagram identifying the infiltration of IL-17-positive cells by immunochemical histology staining in a mouse model injected with PBMCs of a transplantation rejection patient of an embodiment of the present invention.

FIG. 12C is quantification of the number of IL-17-positive cells according to the treatment with the immunosuppressant SD911 in a mouse model injected with normal PBMCs and a mouse model injected with PBMCs of a transplantation rejection patient of an embodiment of the present invention.

### BEST MODES OF THE INVENTION

Hereinafter, embodiments of the present invention will be described in detail with reference to the accompanying drawings. In the following descriptions, the detailed descriptions on the technology known to those skilled in the art will be omitted. In addition, in describing the present invention, when it is determined that the specific description of related known functions or configurations unnecessarily obscure the gist of the present invention, the detailed description therefor may be omitted. Further, terminologies used herein are terms used to properly represent preferred embodiments of the present invention. It may vary depending on the intent of users or operators, or custom in the art to which the present invention belongs.

Accordingly, the definitions of these terms should be based on the contents throughout this specification. In the entire specification, when a part is referred to as “comprising” a component, it means that it may further include other components without excluding other components unless specifically described otherwise.

Hereinafter, the present invention will be described in more detail with reference to the drawings.

An embodiment of the present invention provides a compound represented by Formula 1 below or a pharmaceutically acceptable salt thereof:

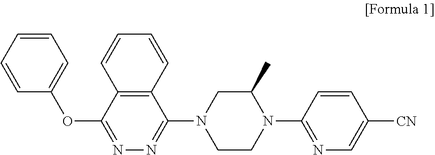 [SMILES: C[C@@H]1CN(c2nnc(Oc3ccccc3)c3ccccc23)CCN1c1ccc(C#N)cn1]

The compound may be synthesized through a process as shown in the following reaction formula, but is not limited thereto:

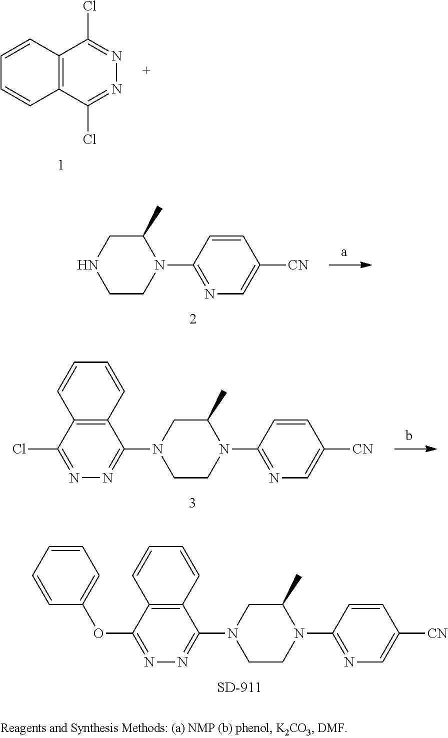 [SMILES: Clc1nnc(Cl)c2ccccc12 ; C[C@@H]1CNCCN1c1ccc(C#N)cn1 ; C[C@@H]1CN(c2nnc(Cl)c3ccccc23)CCN1c1ccc(C#N)cn1 ; C[C@@H]1CN(c2nnc(Oc3ccccc3)c3ccccc23)CCN1c1ccc(C#N)cn1]

The pharmaceutically acceptable salt may include an acid addition salt formed with a pharmaceutically acceptable free acid, and as the free acid, an organic acid and an inorganic acid may be used. Organic acids include citric acid, acetic acid, lactic acid, tartaric acid, maleic acid, fumaric acid, formic acid, propionic acid, oxalic acid, trifluoroacetic acid, benzoic acid, gluconic acid, metasulfonic acid, glycolic acid, succinic acid, 4-toluenesulfonic acid, glutamic acid, and aspartic acid, but are not limited thereto. In addition, inorganic acids include, but are not limited to, hydrochloric acid, bromic acid, sulfuric acid, and phosphoric acid.

According to an embodiment of the present invention, the compound may have a kidney protective efficacy, but is not limited thereto.

According to an embodiment of the present invention, the compound may have an ability to inhibit SIP lyase activity, but is not limited thereto.

In addition, an embodiment of the present invention provides an immunosuppressant including the compound as an active ingredient.

Conventional immunosuppressants such as tacrolimus have kidney toxicity and thus have a problem in long-term administration, but the novel compound of an embodiment of the present invention has kidney protective efficacy, and thus may be used as an immunosuppressant without side effects, but is not limited thereto.

In addition, an embodiment of the present invention relates to a pharmaceutical composition for preventing or treating an immune disease, including a compound or a pharmaceutically acceptable salt thereof of an embodiment of the present invention as an active ingredient.

In the present invention, the “immune diseases” mean diseases in which components of the mammalian immune system cause, mediate, or contribute to the pathological conditions of the mammals. Further, the immune diseases may include all of the diseases in which simulation, or the stop of the immune response has a compensating effect on the progression of the diseases, and in the present invention, may include diseases caused by hypersensitive immune responses. Examples of the immune diseases may include autoimmune diseases; inflammatory diseases; or the like but are not limited thereto.

Further, in all normal subjects, one of the most important features has ability capable of recognizing, responding, and removing non-self-antigens without harmfully responding to self-antigen substances. As such, a non-response to the self-antigen of the living body is called immunologic unresponsiveness or tolerance. However, a disease caused by such a process that when a problem in inducing or continuously maintaining the self-tolerance occurs, the immune response to the self-antigen occurs, and thus a phenomenon in which the self-antigen attacks its own tissue occurs is called an “autoimmune disease.”

The immune disease that may be prevented and treated in the present invention may include rheumatoid arthritis, Behcet's disease, multiple myositis or skin myositis, autoimmune hematocytopenia, autoimmune myocarditis, atopic dermatitis, asthma, primary cirrhosis, dermatomyositis, Goodpasture syndrome, autoimmune meningitis, sjogren's syndrome, lupus, Addison's disease, alopecia areata, ankylosing myelitis, autoimmune hepatitis, autoimmune mumps, Crohn's disease, insulin-dependent diabetes, dystrophic epidermolysis bullosa, epididymitis, glomerulonephritis, Graves' disease, Guillain-Barre syndrome, Hashimoto's disease, hemolytic anemia, multiple sclerosis, myasthenia gravis, pemphigus vulgaris, psoriasis, rheumatic fever, sarcoidosis, scleroderma, spinal arthrosis, thyroiditis, vasculitis, vitiligo, myxedema, pernicious anemia, mitochondrial-related syndromes, ulcerative colitis, and the like, but is not limited thereto.

The composition according to the present invention may contain a pharmaceutically effective amount of the compound or extract alone or may contain one or more pharmaceutically acceptable carriers, excipients, or diluents. In the above, the pharmaceutically effective amount refers to an amount sufficient to prevent, alleviate, and treat symptoms of diseases.

Further, the above “pharmaceutically acceptable” generally means a composition which does not cause an allergic reaction such as gastroenteric trouble and dizziness or a similar reaction thereto when being physiologically acceptable and administrated to the human body. Examples of the carriers, the excipients, and the diluents may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. Further, a filler, an anti-coagulant, a lubricant, a wetting agent, a flavoring, an emulsifier, a preservative, and the like may be additionally included.

Further, the composition of an embodiment of the present invention may be formulated by using a known method in the art so as to provide rapid, sustained, or delayed release of an active component after being administrated to a mammal. The formulation may be in the form of a powder, granules, a tablet, an emulsion, a syrup, an aerosol, a soft or hard gelatin capsule, a sterile injection solution, and a sterile powder.

Further, the administration amount of an active ingredient of the composition of an embodiment of the present invention may be properly selected according to various factors such as an administration route, an age, a gender, and a body weight of a patient, and the severity of the patient. The composition according to an embodiment of the present invention may be administrated by combining a known compound having an effect of preventing, improving, or treating the symptoms of osteoarthritis.

A suitable dosage of the pharmaceutical composition of the present invention may vary depending on factors such as a preparation method, administration method, age, body weight, sex, pathological condition of a recipient, food, administration time, administration route, excretion speed, reaction susceptibility, etc. The preferred daily dosage of the pharmaceutical composition of an embodiment of the present invention is 1×10³ to 1×10¹² cells/kg.

In the composition of an embodiment of the present invention, the compound may be included in a concentration of 1 to 20 μM, for example, 1 to 15 μM, 1 to 10 μM, 1 to 5 μM, 2 to 20 μM, 5 to 20 μM, or 10 to 20 μM but is not limited thereto.

In addition, an embodiment of the present invention provides a method for preventing or treating an immune disease, in which the method includes administering to a subject an effective amount of a compound represented by Formula 1 or a pharmaceutically acceptable salt thereof.

The treatment method of an embodiment of the present invention includes administering the pharmaceutical composition to a subject in a therapeutically effective amount. The specific therapeutically effective amount for any particular subject may vary depending on various factors well known in the medical art and other factors including the kind and degree of the response to be achieved, whether other agents are used therewith or not in some cases, specific compositions, the patient's age, body weight, health conditions, gender, and diet, the time and route of administration, the secretion rate of the composition, the time period of therapy, or other drugs used in combination or coincidentally with the specific composition. Therefore, the effective amount of the composition suitable for the purpose of the present invention is preferably determined in consideration of the matters as described above.

The subject is applicable to any mammal. The mammal includes livestock such as cows, pigs, sheep, horses, dogs, and cats as well as humans and primates.

In addition, an embodiment of the present invention provides a pharmaceutical composition for preventing or treating transplantation rejection or transplantation rejection disease, including the compound as an active ingredient.

According to an embodiment of the present invention, the transplantation rejection is one or more transplantation rejections selected from the group consisting of cells, blood, tissues, and organs, and preferably organ transplantation rejection, but is not limited thereto.

According to an embodiment of the present invention, the transplantation rejection is one or more transplantation rejections selected from the group consisting of bone marrow transplantation, heart transplantation, corneal transplantation, bowel transplantation, liver transplantation, lung transplantation, pancreas transplantation, kidney transplantation, and skin transplantation, and preferably kidney transplantation rejection, but not limited thereto.

According to an embodiment of the present invention, the transplantation rejection disease may be graft-versus-host disease (GVHD) or post transplantation late and chronic solid organ rejection, but not limited thereto.

In addition, an embodiment of the present invention provides a method for preventing or treating transplantation rejection or transplantation rejection disease, in which the method includes administering to a subject an effective amount of a compound represented by Formula 1 or a pharmaceutically acceptable salt thereof.

In addition, an embodiment of the present invention provides a humanized transplantation rejection animal model in which immunodeficient mice are administered with PBMCs (peripheral blood mononuclear cells) derived from transplantation rejection patients.

As used herein, the term “transplantation rejection” refers to a reaction that, recognizing the transplanted tissue as non-self after the transplantation, the recipient's immune system attacks and removes the transplanted tissue or organ. The most important factor involved in transplantation rejection is the major histocompatibility complex (MHC), and the minor histocompatibility complex is also known to be related. Both cell-mediated immune response and humoral immune response are involved in the rejection reaction. Cell-mediated reactions are caused by CD4 T cell-type II MHC molecules or CD8 T cell-type I MHC molecules through the encounter of the recipient's lymphocytes with the donor's MHCs. Activated T cells secrete cytokines, increase blood vessel permeability, and bring about infiltration of monocytes such as macrophages, thereby resulting in damage to micro-vessels, tissue ischemia, and destruction of transplanted tissues and cells.

The transplantation rejection is one or more transplantation rejections selected from the group consisting of cells, blood, tissues, and organs. Preferably, the transplantation rejection is one or more selected from the group consisting of rejections of bone marrow transplantation, heart transplantation, corneal transplantation, bowel transplantation, liver transplantation, lung transplantation, pancreas transplantation, kidney transplantation, and skin transplantation, but is not limited thereto.

According to an embodiment of the present invention, the PBMC derived from the transplantation rejection patient may be administered at a concentration of 1 to 5×10⁶, preferably 5×10⁶, but is not limited thereto.

In an embodiment of the present invention, the derived mouse is not limited, and general laboratory mice, immunodeficient mice, and the like may be used.

As used herein, the term “immunodeficient mouse” refers to a mouse characterized by one or more of: a lack of functional immune cells, such as T cells and B cells, a DNA repair defect; a defect in the rearrangement of genes encoding antigen-specific receptors on lymphocytes; and a defect of immune functional molecules such as IgM, IgG1, IgG2a, IgG2b, IgG3, and IgA. In an embodiment, immunodeficient mice may be characterized by one or more deficiencies in a gene involved in immune function, such as Rag1 and Rag2 (Oettinger et al., Science. 248:1517-1523, 1990; and Schatz et al., Cell. 59:1035-1048, 1989) Immunodeficient mice may have any of these or other defects which result in abnormal immune function in the mice.

Particularly useful immunodeficient mouse strains are NOD.Cg-Prkd^{scid}Il2rg^{tm1 Wj1}/SzJ, commonly referred to as NOD scid gamma (NSG) mice, described in detail in Shultz et al., J. Immunol., 174:6477-6489, 2005; and NOD.Cg-Rag1^{tm1Mom}Il2rg^{tm1 Wj1}/SzJ, commonly referred to as NRG mice (Shultz et al., Clin. Exp. Imnunol., 154(2):270-284, 2008).

According to an embodiment of the present invention, in the animal model, creatinine in serum may be increased compared to the reference value of the control group.

According to an embodiment of the present invention, in the animal model, human CD4-positive cells may be increased compared to the reference value of the control group.

According to an embodiment of the present invention, in the animal model, the infiltration of the inflammatory cytokine IL-17 into tissue cells may be increased compared to the reference value of the control group.

According to an embodiment of the present invention, the transplantation rejection may be rejection by a kidney transplantation.

In addition, an embodiment of the present invention provides a method for constructing a humanized transplantation rejection animal model, in which the method includes injecting PBMCs isolated from transplantation rejection patients into immunodeficient mice.

According to an embodiment of the present invention, the injecting of the PBMCs may be performed 1 to 5 times for 0 to 4 weeks.

In addition, an embodiment of the present invention provides a method for screening a transplantation rejection therapeutic agent, in which the method includes treating a candidate substance in the humanized transplantation rejection animal model.

As used herein, the term “immunosuppressant” is a drug that reduces or inhibits the body's immune system activity, and is a drug that is largely classified as steroids, cell proliferation inhibitors, antibody preparations, drugs acting on immunophilin, mycophenolate, and tumor necrosis factor (TNF-α) inhibitors.

As such, immunosuppressants, which have been used for a transplantation surgery and administered to many patients with immune diseases may cause various side effects in the body, and particularly, as for a patient after surgery such as transplant, there is no alternative but to prescribe an immunosuppressant to suppress an immune rejection response even after considering that side effects inevitably occur. Hence, in the case of transplantation rejection, it is important to screen for an appropriate immunosuppressant according to the immune system of a transplanted patient.

According to an embodiment of the present invention, the candidate substance may be an immunosuppressant, and the immunosuppressant may be any one selected from the group consisting of SD911, tacrolimus, cyclosporine A, prednisolone, methylprednisolone, deflazacort, mycophenolic acid, azathioprine, mizoribine, sirolimus, and everolimus.

According to an embodiment of the present invention, the SD911 may be represented by Formula 1.

According to an embodiment of the present invention, the candidate substance may reduce creatinine in serum.

According to an embodiment of the present invention, the candidate substance may reduce human CD4-positive cells.

According to an embodiment of the present invention, the candidate substance may be to reduce the infiltration of the inflammatory cytokine IL-17 into tissue cells.

### MODES OF THE INVENTION

Hereinafter, the present invention will be described in detail by way of Examples. However, these Examples are for explaining the present invention in more detail, and the scope of the present invention is not limited to these Examples.

### <Preparation Example 1> Preparation of SD911 Compound

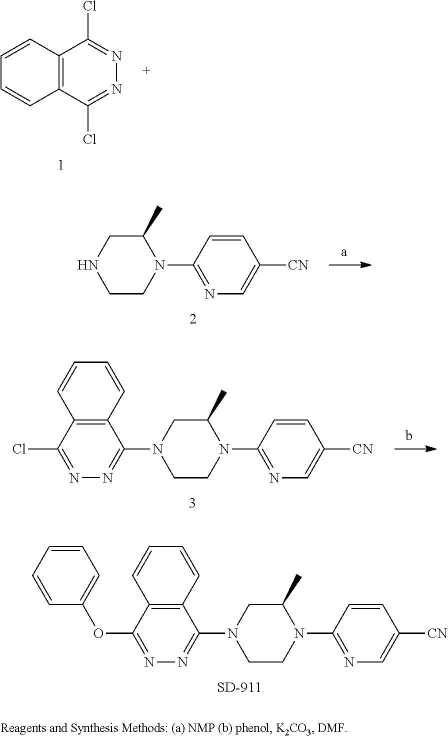 [SMILES: Clc1nnc(Cl)c2ccccc12 ; C[C@@H]1CNCCN1c1ccc(C#N)cn1 ; C[C@@H]1CN(c2nnc(Cl)c3ccccc23)CCN1c1ccc(C#N)cn1 ; C[C@@H]1CN(c2nnc(Oc3ccccc3)c3ccccc23)CCN1c1ccc(C#N)cn1]

### 6-((R)-4(1-chlorophthalazin-4-yl)-2-methylpiperazin-1-yl)pyridine-3-carbonitrile (3)

1,4-diclonaphthalein (494 mg, 2.48 mmol) was added to Compound No. (2) (502 mg, 2.48 mmol) in an NMP (3.5 ml) solution. After 24 hours, the reaction mixture was diluted with ethyl acetate, and the organic phases were combined and washed with water, and concentrated under reduced pressure. The residue was purified by flash column chromatography on silica gel (ethyl acetate:N-hexane=1:2) to obtain compound No. (3) (49.4 mg, yield=5.46%). MS (ESI) [M+H]+ 365

### 6-((R)-2-methyl-4-(1-phenoxyphthalazin-4-yl)piperazin-1-yl)pyridine-3-carbonitrile (SD-911)

K₂CO₃ (16 mg, 0.1161 mmol) was added to compound No. (3) (15.7 mg, 0.043 mmol) and phenol (5.3 mg, 0.059 mmol) in a DMF solvent (0.05 ml). After 24 hours, the reaction mixture was diluted with ethyl acetate, and the organic phases were combined and washed with water, and concentrated under reduced pressure. The residue was purified by flash column chromatography on silica gel (ethyl acetate:N-hexane=1:2) to obtain a compound, which was named “SD-911” (14.5 mg, yield=80%).

¹H NMR (600 MHz, CDCl₃) δ (ppm)=8.43 (d, J=6.0 Hz, 1H), 8.40 (t, J=6.0 Hz, 1H), 8.15 (m, 1H), 7.92 (m, 2H), 7.64 (m, 1H), 7.42 (m, 2H), 7.28 (d, J=12 Hz, 2H), 7.23 (m, 1H), 6.64 (d, J=6.0 Hz, 1H), 4.74 (bs, 1H), 4.32 (d, J=18 Hz, 1H), 3.80 (d, J=18 Hz, 1H), 3.67 (d, J=18 Hz, 1H), 3.54 (m, 1H), 3.29 (m, 1H), 3.20 (d, J=6.0 Hz, 1H), 3.15 (m, 1H), 1.52 (d, J=6.0 Hz, 3H); HMS (ESI) [M+H]+ 423.

### <Example 1> Cell Line Culture

The human kidney-2 cell line used in this experiment was purchased from ATCC (Manassas, Va., USA) and used. These cells were cultured in Dulbecco's modified Eagle's medium (DMEM; Wisent) containing 10% fetal bovine serum (FBS; Wisent, St. Bruno, Que, Canada), 100 U/mL penicillin, and 100 mg/mL streptomycin (Wisent) in 5% CO₂, 37° C. incubator.

### <Example 2> Cell Viability Investigation

After 80% seeding of HK-2 cells, 24 hours later, tacrolimus was treated at a concentration of 60 to 70 μg/ml and SD911 was treated at a concentration of 1 to 20 μM and reacted for 3 to 12 hours. In the case of cells isolated as a single cell from mouse spleen tissue, after seeding 2×10⁵/cell/200 μl in a 96-well plate in triplicate, each SD911 compound was treated with 1 to 20 μM and reacted for 3 days. Thereafter, the cell counting kit-8 (CCK-8, Dojindo molecular, Rockville, Mass., USA) reagent was treated for 2 hours, and then measured and analyzed at absorbance of 450 nm.

<2-1> Identification of Cell Viability of SD911 in HK-1 Cells

The cytoprotective effect of an SD911 compound was investigated under HK-2 cytotoxicity induction by tacrolimus (Tac). As can be seen in FIG. 1, it was identified that compared to Nil, SD911 at concentrations of 1, 5, 10, and 20 μM did not have its own drug toxicity. When Nil was 100%, tacrolimus (Tac) showed a survival rate of 56.7%. However, when 1, 5, and 10 μM of SD911 were treated in combination with tacrolimus (Tac), although the survival rates were significantly improved to 81.7%, 81.8%, and 72.0%, respectively, 20 μM of SD911 showed a lower survival rate of 37.4% compared to tacrolimus (Tac) alone (^{$}P<0.05 vs. Nil; ^{#}P<0.05 vs. Tac).

<2-2> Toxicity Test of SD911 on Mouse Splenocytes

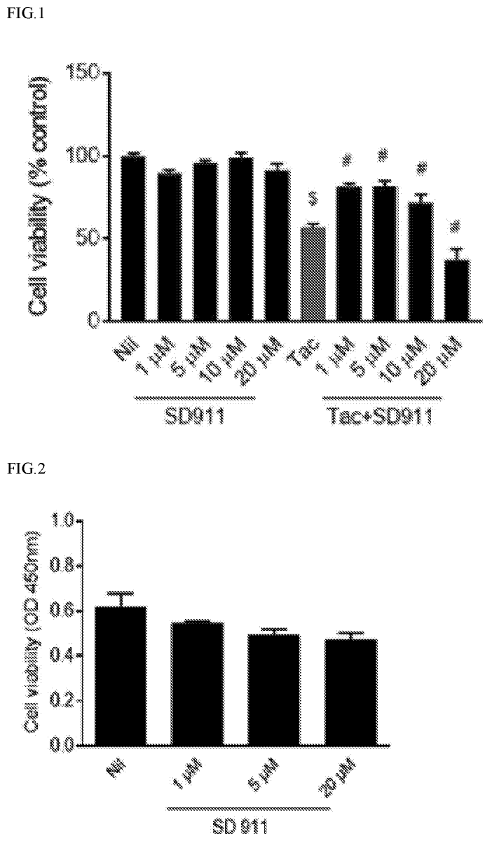

As can be seen in FIG. 2, as a result of conducting a toxicity investigation on immune cells of the SD911 compound (1, 5, 20 μM), there was no significant difference compared to the Nil group.

### <Example 3> Measurement of Reactive Oxygen Species (ROS)

After 80% seeding of HK-2 cells, 24 hours later, tacrolimus was treated at a concentration of 60 to 70 μg/ml and SD911 was treated at a concentration of 1 to 20 μM and reacted for 3 to 12 hours, and then 10 μM of DCF-DA (Molecular probes, Carlebad, Calif., USA) was reacted at 37° C. for 1 hour. Then, cells were harvested and analyzed with 0.05% Trypsin and 0.53 mM EDTA. For analysis, aggregated or broken cells were removed using FSC (forward side scatter) and SSC (side scatter) scatter plots (dot plot) using a FACS Calibur (BD bioscience, San Jose, Calif., USA), and the fluorescence fraction was analyzed in the FL-1H channel (Excitation 488 nm, Emission 513 to 535 nm).

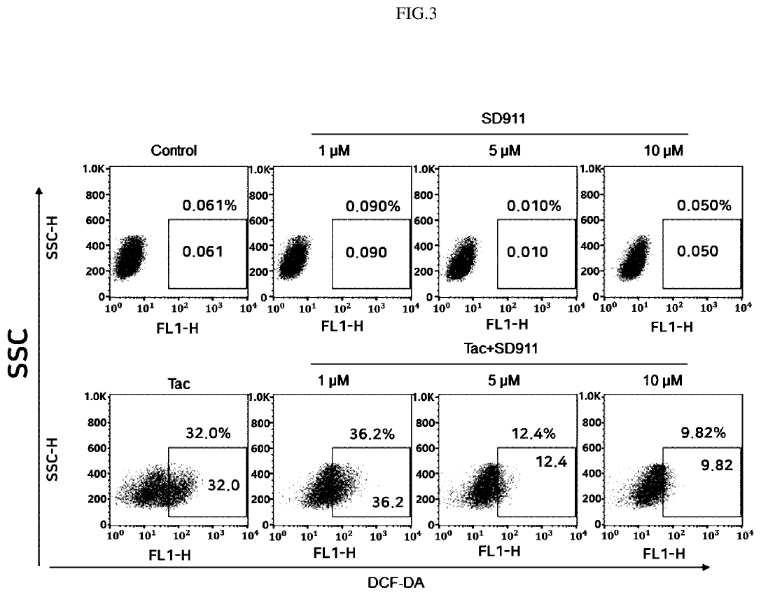

Based on the cell viability identification results of Example 1, the ROS production inhibitory effect on 1, 5, and 10 μM of SD911 was investigated. As can be seen in FIG. 3, for SD911 alone at 1, 5, and 10 μM, the fluorescence fraction of DCF-DA, a reagent for detecting ROS, had almost no difference from that of Nil, a control. Upon induction of HK-2 cytotoxicity by tacrolimus (Tac), ROS was increased at a rate of 31.6%, and at 5 and 10 μM, it was significantly decreased to 13.7% and 9.3%, respectively. However, 1 μM of the SD911-combined group was rather 36.5%, not significantly different from the tacrolimus (Tac) group (^{#}P<0.05 vs. Tac).

### <Example 4> ELISA Analysis

Cell culture for ELISA was coated with 250 ul of mouse anti-CD3 antibody diluted in ccPBS with 0.5 μg/ml in a 24-well plate as many as the number of conditions. After incubation for 2 hours, the supernatant was removed and the cells were seeded at 1×10⁶/cells/1 ml and then treated with each drug. After culturing for 3 days, only the cell culture medium was removed and used as IL-10 and IL-17 ELISA samples.

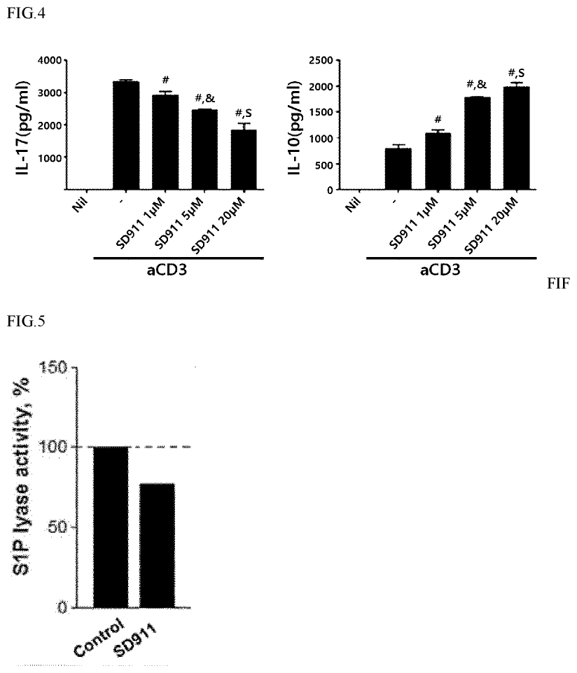

As can be seen in FIG. 4, it was identified that the SD911 drug decreased IL-17 and increased IL-10 in a concentration-dependent manner compared to the group not treated with the SD911 compound under anti-CD3 immune activity (^{#}P<0.05 vs. aCD3 only; ^{&}P<0.05 vs. 1 μM SD911; ^{$}P<0.05 vs. 5 μM SD911).

### <Example 5> Statistical Analysis Method

For the analysis results, the PRISM software (version 7.03 for Windows, GraphPad Software, LaJolla, Calif., USA) statistical program was used. For multiple comparisons, 1-way ANOVA with Bonferroni's post hoc test was used to test the average value between each group. The P value less than or equal to 0.05 was interpreted as statistically significant.

### <Example 6> SPL Inhibitory Effect of SD911 Compound

After homogenizing the liver tissue of an experimental animal in a lysis buffer, protein quantification was performed to secure the concentration, and a mixed solution was prepared under the conditions shown in Table 1 below and reacted at 37° C. for 1 hour. 20 ul 0.1 nmol/ul heptadecanal (C17) and 180 ul MeOH were added, and a standard was prepared as shown in Table 2 below, then measured and analyzed by HPLC.

TABLE 1

| | Control | | Control | | 5 uM | |
|---|---|---|---|---|---|---|
| | (−) | | (+) | | inhibitor | |
| Reaction buffer | 140 | ul | 140 | ul | 140 | ul |
| 4 mg/ml BSA | 30 | ul | X | | | |
| C18:0 SalP (in BSA) | X | | 30 | ul | 30 | ul |
| D.D.W | 10 | ul | 10 | ul | 10 | ul |
| DMSO | 10 | ul | 10 | ul | | |
| 100 uM | X | | X | | 10 | ul |
| Inhibitor | | | | | | |
| (in DMSO) | | | | | | |
| (720 ug) | 10 | ul | 10 | ul | 10 | ul |
| Liver lysate | | | | | | |
| Total | 200 | ul | 200 | ul | 200 | ul |

TABLE 2

| | 0 nmol | | 0.5 nmol | | 1 nmol | | 2 nmol | |
|---|---|---|---|---|---|---|---|---|
| | C16 | | C16 | | C16 | | C16 | |
| Rxn buffer | 200 | ul | 200 | ul | 200 | ul | 200 | ul |
| 0.1 nmol/ul | 0 | ul | 5 | ul | 10 | ul | 20 | ul |
| Hexadecanal (C16) | | | | | | | | |
| 0.1 nmol/ul | 20 | ul | 20 | ul | 20 | ul | 20 | ul |
| heptadecanal (C17) | | | | | | | | |
| MeOH | 200 | ul | 175 | ul | 170 | ul | 160 | ul |

| | |
|---|---|
| Total | 400 ul |

As a result, as can be seen in FIG. 5, compared to the control, the SD911 compound, which is an SPL (S1P lyase) inhibitor, showed about 30% reduced S1P (Sphingosine 1-phosphate) lyase activity, identifying that SIP lyase activity inhibitory power was present. As such, it may be expected that the SD911 compound has an ability to inhibit SPL activity, and thus may be applied to the treatment of diseases related to abnormal immune activity.

### <Example 7> Identification of Kidney Protective Effect

In HK-1 cells, the effect of SD911 on ROS production inhibitory ability and apoptosis reduction was identified. Based on the cell viability test results of Example 2 (FIG. 2), inhibitory effects of SD911 on ROS production were investigated at 1, 5, and 10 μM of SD911.

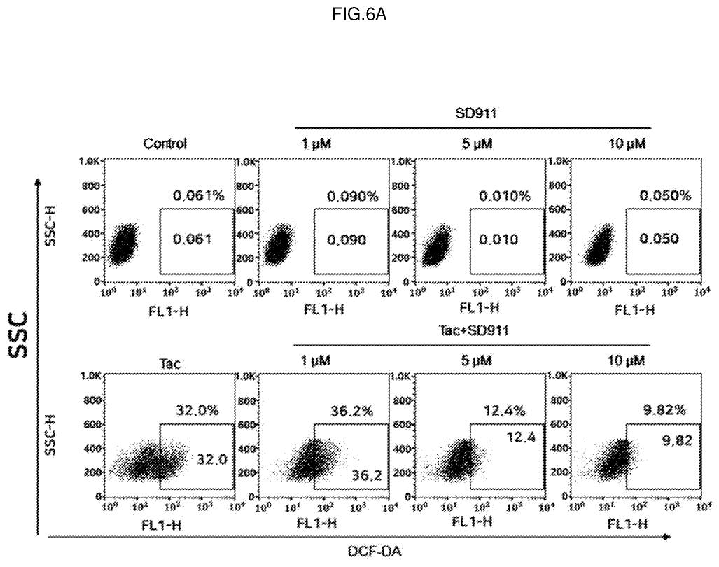

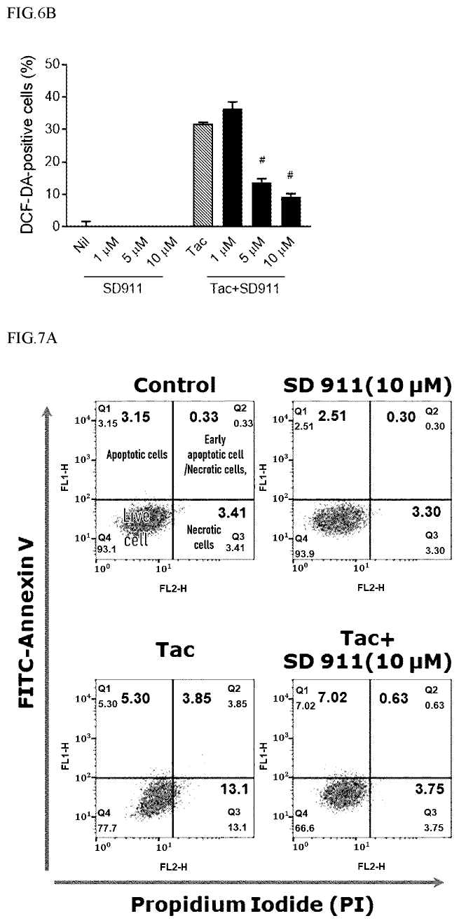

As a result, as can be seen in FIGS. 6A and 6B, the fluorescence fraction of DCF-DA, a reagent for detecting ROS for SD911 compound alone at 1, 5, and 10 μM, had almost no difference from that of Nil, a control. Upon induction of HK-2 cytotoxicity by Tac, ROS was increased at a rate of 31.6%, and at 5 and 10 μM, it was significantly decreased to 13.7% and 9.3%, respectively. However, 1 μM of the SD911-combined group was rather 36.5%, not significantly different from the Tac group (^{#}P<0.05 vs. Tac).

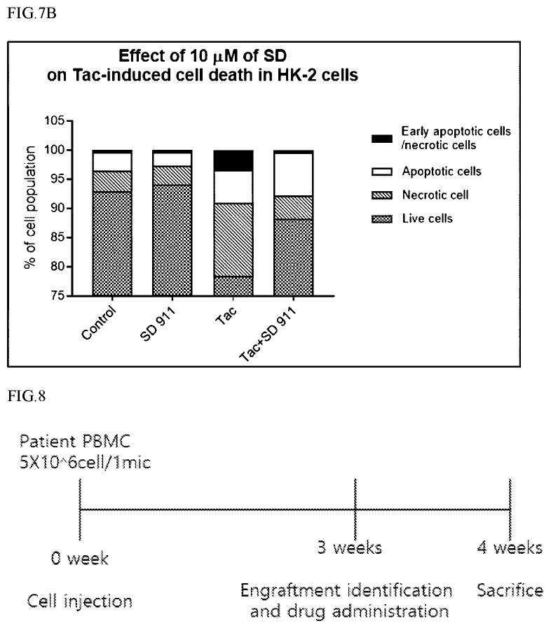

In addition, in order to investigate the effect of SD911 on Tac-induced apoptosis, an analysis was performed with drugs of P1 and Annexin-V. As a result, in the combined group of Tac and SD911 (10 μM), it was identified that the ratio of necrotic cells, apoptotic cells, and early apoptotic cells & necrotic cells was significantly alleviated compared to Tac alone (FIGS. 7A and 7B).

### <Example 8> Construction of Evaluation Platform for Imitation Avatar Model of Transplantation Rejection Patients

In order to produce the transplantation rejection avatar animal model of an embodiment of the present invention, PBMCs (peripheral blood mononuclear cells) derived from normal or transplantation rejection patients were intravascularly injected into 8 to 10 week old immunodeficient mice (NSG) at 5×10⁶/mice. After 3 weeks, the engraftment of normal or transplantation rejection patients (kidney transplantation rejection patients) cells in the blood was identified in blood cells. Thereafter, the mice were sacrificed 4 weeks after cell transplantation to identify the infiltration of human cells and histological changes in the tissue (FIG. 8).

### <Example 9> Identification of Engraftment of Human Cells in Blood of Transplantation Rejection Patient Imitation Avatar Model

In order to identify that the transplantation rejection patient imitation avatar model in Example 8 above was properly constructed, engraftment of human cells was analyzed through flow cytometry. Specifically, blood was obtained from the transplantation rejection humanized mice of Example 1 (normal PBMC injection group, HC; transplantation rejection patient PBMC injection group, Patient), and then cells that were positive in reaction with human antibodies were analyzed. In addition, blood creatine (SCR) concentration was measured as an index for measuring kidney damage in PBMC-injected mice of normal subjects and transplantation rejection patients. SCR was measured by the quantitative enzymatic colorimetric method (Stanbio laboratory, 0430-120) by isolating serum in animal blood.

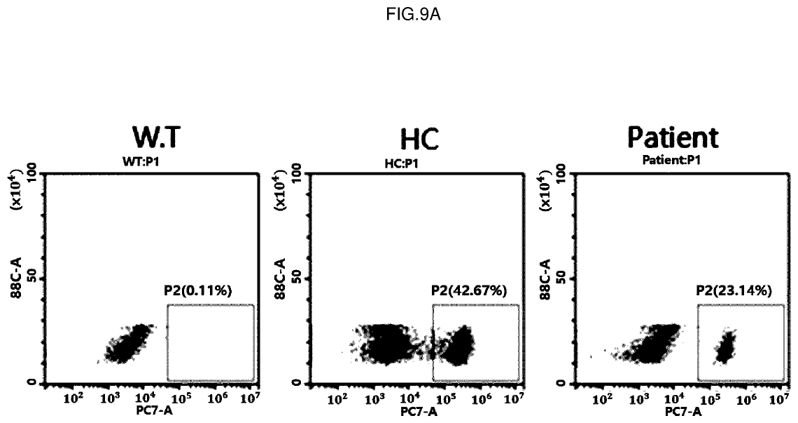

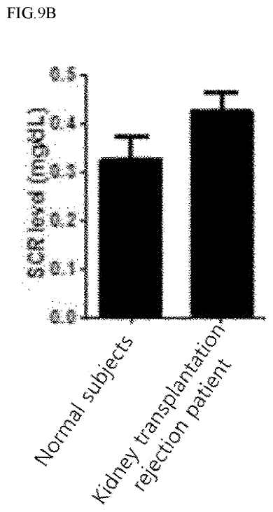

As a result, as shown in FIG. 9A, it was identified that human cells were engrafted by flow cytometry.

In addition, as shown in FIG. 9B, it was identified that the SCR level was higher in the transplantation rejection patient PBMC injection group (kidney transplantation rejection patients) than in the normal PBMC injection group (normal subjects).

### <Example 10> Identification of Immunosuppressant Effect on Transplantation Rejection Patient Imitation Avatar Model

<10-1> Identification of Kidney Tissue Damage Control

Specifically, the transplantation rejection humanized mouse model established in Example 8 above was classified as 1) a normal PBMC injection group, 2) a normal PBMC injection group treated with an immunosuppressant, 3) a transplantation rejection patient PBMC injection group, and 4) a transplantation rejection patient PBMC injection group treated with an immunosuppressant. SD911 was used as the immunosuppressant. Specifically, the animal model of Example 8 above was treated with SD911 at 3 weeks after engraftment of PBMCs. and the same amount of physiological saline was treated as a control group. One week after drug treatment, the mice were sacrificed, the kidneys were excised, and the degree of tissue damage was identified. Specifically. GN score (membranous glomerulonephritis score), which is an indicator that identifies damage to the glomerulus, IN score (renal interstitial nephritis score), which is an indicator of kidney immune cell infiltration, and Vasculitis, which is an indicator that identifies the degree of infiltration of immune cells around blood vessels, were measured, respectively.

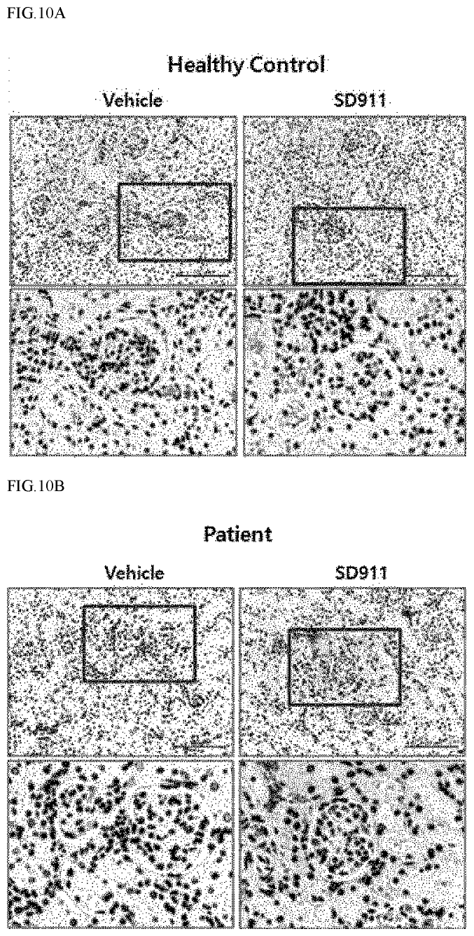

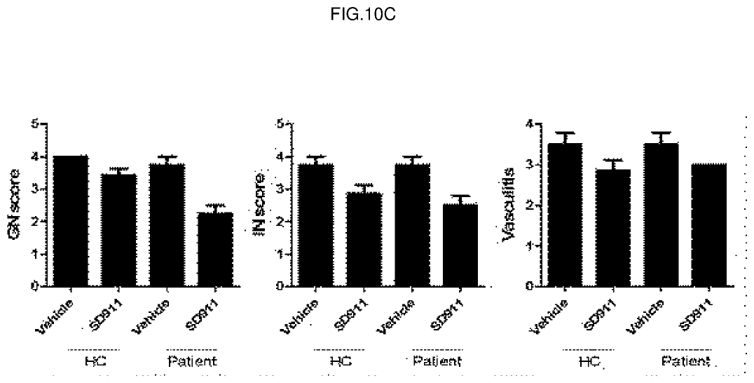

As a result, it was identified that the group injected with transplantation injection patient PBMCs significantly increased damage to kidney tissue than the group injected with normal PBMCs (FIGS. 10A and 10B). In addition, it was identified that when the immunosuppressant SD911 was administered to the group injected with transplantation injection patient PBMCs, the damage to the kidney tissue was reduced (FIGS. 10B and 10C).

<10-2> Identification of Etiological T Cell Infiltration Control in Kidney Tissue

In order to identify whether CD4+T (CD4⁺), a human immune cell subtype, was infiltrated in the kidney tissue excised in Example 10-1, immunohistochemical staining was performed. The excised tissue was fixed with formalin and then embedded in paraffin to produce a 5 μm thick section. In order to observe immune cells in the tissue, immunohistochemical analysis was performed by reacting with human CD4 antibody on the section slide.

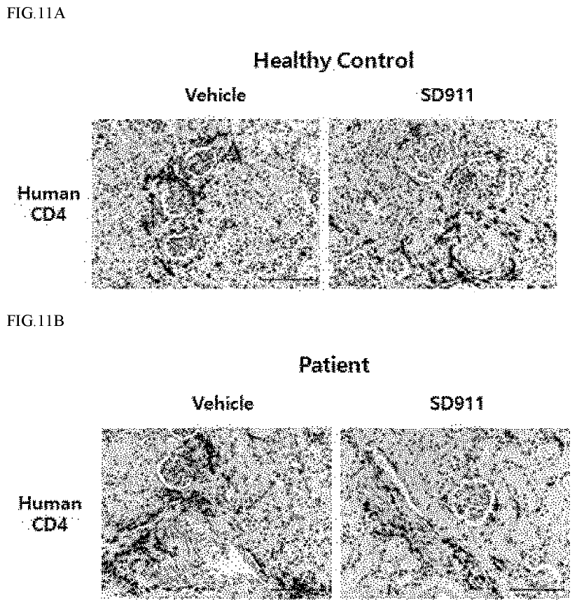

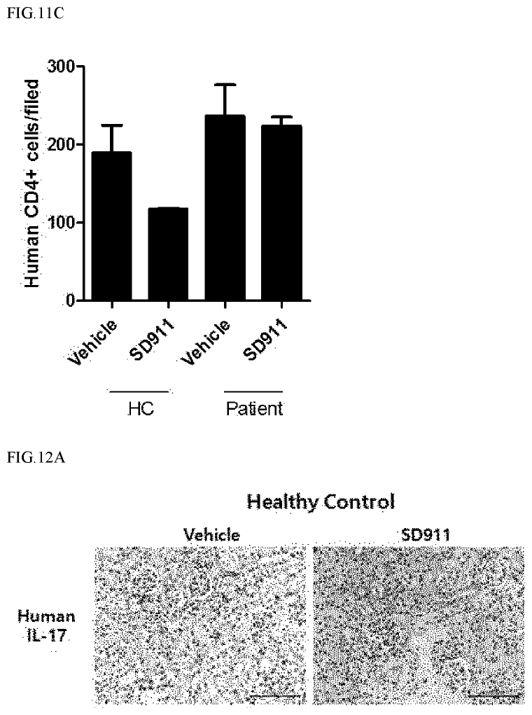

As a result, it was identified that human CD4+ T cells were detected in the mouse kidney tissue injected with PBMCs of normal subjects and transplant rejection patients of Example 8, and that human cells were well engrafted. When SD911 was treated, in the group injected with transplantation injection patient PBMCs, it was identified that the infiltration of CD4-positive cells was significantly reduced and was reduced compared to the group injected with normal PBMCs (FIGS. 11A to 11C).

<10-3> Identification of IL-17 Infiltration Control in Kidney Tissue

In order to identify that IL-17, an inflammatory cytokine in the kidney tissue excised in Example 10-1, was infiltrated, immunohistochemical staining was performed. The excised tissue was fixed with formalin and then embedded in paraffin to produce a 5 μm thick section. In order to observe immune cells in the tissue, immunohistochemical analysis was performed by reacting with human IL-17 antibody on the section slide.

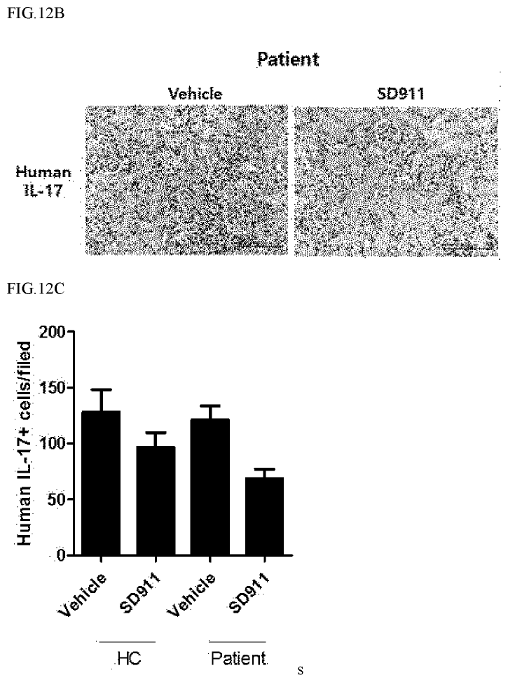

As a result, it was identified that human IL-17 cells were detected in the mouse kidney tissue injected with PBMCs of normal subjects and transplant rejection patients of Example 8, and that human cells were well engrafted. When SD911 was treated, in the group injected with transplantation injection patient PBMCs, it was identified that the infiltration of IL-17 positive cells was significantly reduced and was reduced compared to the group injected with normal PBMCs (FIGS. 12A to 12C).

Accordingly, the novel compound of an embodiment of the present invention has an excellent immunological regulation ability while not exhibiting toxicity in vivo, and has no nephrotoxicity unlike conventional immunosuppressants and rather has a kidney protective effect, and thus can be used for the treatment of immune diseases such as autoimmune diseases caused by abnormal regulation of various immune responses, inflammatory diseases, and transplantation rejection diseases. A transplantation rejection avatar animal model of an embodiment of the present invention was confirmed to be humanized, by confirming an increase in serum creatinine, which is an indicator of transplantation rejection in a patient, an increase in human CD4-positive cells, and infiltration of inflammatory cytokine IL-17 into kidney tissue of the animal model. It has also been confirmed that, when an immunosuppressant is administered, the increased serum creatinine, human CD4-positive cells, and the infiltration of inflammatory cytokine IL-17 are reduced, and thus an animal model, in which the immune status of a patient is reflected, is effectively constructed, and the consequent effect of the immunosuppressant has been confirmed.

1. A compound represented by Formula 1 below or a pharmaceutically acceptable salt thereof:

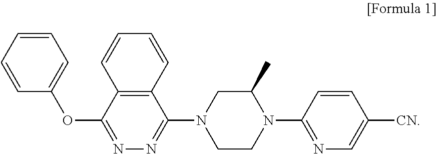 [SMILES: C[C@@H]1CN(c2nnc(Oc3ccccc3)c3ccccc23)CCN1c1ccc(C#N)cn1]

2. The compound or the pharmaceutically acceptable salt thereof of claim 1, wherein the compound has a kidney cell protective effect.

3. The compound or the pharmaceutically acceptable salt thereof of claim 1, wherein the compound inhibits S1P lyase activity.

4. An immunosuppressant comprising the compound of claim 1 as an active ingredient.

5. A pharmaceutical composition for preventing or treating an immune disease, comprising the compound of claim 1 as an active ingredient.

6. The pharmaceutical composition of claim 5, wherein the immune disease includes an autoimmune disease.

7. The pharmaceutical composition of claim 5, wherein the compound has kidney protective efficacy.

8. The pharmaceutical composition of claim 5, wherein the compound has an ability to inhibit S1P lyase activity.

9. A method for preventing or treating an immune disease, the method comprising administering to a subject an effective amount of a compound represented by Formula 1 or a pharmaceutically acceptable salt thereof:

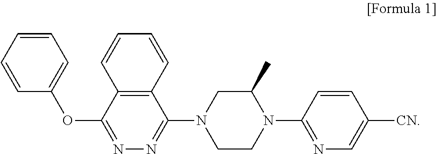 [SMILES: C[C@@H]1CN(c2nnc(Oc3ccccc3)c3ccccc23)CCN1c1ccc(C#N)cn1]

10. A pharmaceutical composition for preventing or treating transplantation rejection or transplantation rejection disease, comprising the compound of claim 1 as an active ingredient.

11. The pharmaceutical composition of claim 10, wherein the transplantation rejection is one or more transplantation rejections selected from the group consisting of cells, blood, tissues, and organs.

12. The pharmaceutical composition of claim 11, wherein the transplantation rejection is one or more transplantation rejections selected from the group consisting of bone marrow transplantation, heart transplantation, corneal transplantation, bowel transplantation, liver transplantation, lung transplantation, pancreas transplantation, kidney transplantation, and skin transplantation.

13. The pharmaceutical composition of claim 10, wherein the transplantation rejection disease is graft-versus-host disease (GVHD) or post transplantation late and chronic solid organ rejection.

14. A method for preventing or treating transplantation rejection or transplantation rejection disease, the method comprising administering to a subject an effective amount of a compound represented by Formula 1 below or a pharmaceutically acceptable salt thereof: 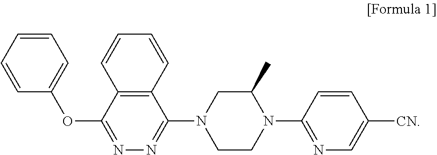 [SMILES: C[C@@H]1CN(c2nnc(Oc3ccccc3)c3ccccc23)CCN1c1ccc(C#N)cn1]
